(19) United States
(12) Patent Application Publication
(10) Pub. No.: US 2011/0300094 A1
(43) Pub. Date: Dec. 8, 2011

(54) Silicone Oil-In-Water Emulsions

(76) Inventor: Tatiana Dimitrova, Braine-L'Alleud (BE)

(21) Appl. No.: 13/139,968

(22) PCT Filed: Dec. 15, 2009
(86) PCT No.: PCT/EP2009/067147

§ 371 Date: Aug. 29, 2011

(30) Foreign Application Priority Data
Dec. 15, 2008 (GB) GB 0822823.1
Dec. 15, 2009 (EP) PCT/EP2009/067147

(51) Int. Cl. A61K 8/92; A61K 8/64; C09K 3/00; A61Q 19/00; A61Q 5/02; A61Q 19/10; A61Q 5/12; A61K 47/42; C11D 3/60

(52) U.S. Cl. 424/70.122; 514/773; 514/775; 512/2; 516/55; 510/527; 424/70.12; 514/63

(57) ABSTRACT

A silicone oil-in-water emulsion comprising 3 to 90% by volume of a silicone oil phase, characterized in that the emulsion is stabilized by a protein and/or peptide present at 0.25 to 20% by weight of the emulsion. The emulsion contains less than 25% by weight and preferably zero % by weight of non-polymeric amphiphilic surfactant based on the weight of protein or peptide. A process for preparing the emulsion is also described.

### CROSS REFERENCE TO RELATED APPLICATIONS

None

### TECHNICAL FIELD

This invention relates to silicone oil-in-water emulsions comprising droplets of silicone oil dispersed in a continuous aqueous phase. Such emulsions are used in many products to enhance the products with the benefits of silicones. For example, silicones (organopolysiloxanes) are present in many hair shampoos and other hair care products to enhance the shine and healthy appearance of the hair and are also present in skin care products such as shower gels to enhance the smooth feel of the skin. Silicones are also present in laundry products such as rinse cycle fabric softeners to give a soft feel to fabrics.

### BACKGROUND

Silicone oil-in-water emulsions require an additive to stabilize the emulsion, that is to prevent the droplets of silicone oil from coalescing into a continuous oil phase. The additives used are surfactants, that is amphiphilic molecules comprising a hydrophobic portion and a hydrophilic portion. The surfactants are generally non-polymeric and may be anionic, cationic, nonionic or amphoteric. Examples of such surfactants suitable for silicon oil-in-water emulsions are given in many published patents, for example in WO-02/42360-A2.

There are however some products in which the usual amphiphilic surfactants can cause problems. In particular, more and more end users are developing allergies and therefore need products that do not contain potentially irritant and potentially sensitizing molecules as surfactants. This is particularly true for preparations designed for young children, such as baby wipes or baby shampoo. Amphiphilic surfactants may also generate a lot of foam when disposed of in sewage, and are unfriendly to aquatic life. In some applications such as food and cosmetics the formulation freedom and choice of surfactants are limited by legislation.

GB1154256 describes a tablettable alkylpolysiloxane composition which is prepared from an emulsion comprising a liquid alkylpolysiloxane in an aqueous solution of skimmed milk powder.

JP58-063750 describes a polydimethylsiloxane in water emulsion stabilized by protein, protein hydrolysate and one or more non-polymeric amphiphilic surfactant(s). The water phase in all cases contains a polyhydroxy compound. However as the surfactant is more surface active they will adsorb preferentially on the interface and as such will tend to displace the proteins from the interface. When protein is used alone as the stabilizer and the water phase contains a polyhydroxy compound (for example propylene glycol, sorbitol or glucose) the polyhydroxy compound is expected to modify the solvent quality with respect to protein (a polymer) thus facilitating emulsion formation. For example in the case of propylene glycol the surface tension of propylene glycol is about 40 mN/m at room temperature whilst the surface tension of water is 72 mN/m. The lower surface tension of the propylene glycol is also believed to facilitate the emulsification.

### SUMMARY

A silicone oil-in-water emulsion comprising 3 to 90% by volume of a silicone oil phase, characterized in that the emulsion is stabilized by a protein and/or peptide present at 0.25 to 20% by weight of the emulsion and that the emulsion contains less than 25% by weight of non-polymeric amphiphilic surfactant based on the weight of protein or peptide.

In a process for the preparation of such an emulsion, the silicone oil and the protein or peptide are mechanically mixed under high shear.

### DETAILED DESCRIPTION

The silicone oil phase generally comprises a fluid organopolysiloxane composition. The fluid organopolysiloxane composition can for example have a bulk viscosity of at least 1 or 5 up to 1000000 centiStokes or even up to 20000000 centiStokes (1 or 5 mm²/sec up to 1 or even 20 m²/sec.) The fluid organopolysiloxane can for example be a substantially linear polydiorganosiloxane, for example of viscosity 100 to 60000 centiStokes such as polydimethylsiloxane although branched and/or cyclic polysiloxanes can also be emulsified. The organopolysiloxane fluid may be a non-reactive fluid, for example a linear polydimethylsiloxane tipped with trimethylsiloxy units, or may be an organopolysiloxane fluid having reactive groups.

The silicone oil phase can be a mixture of two or more organopolysiloxanes. For example the silicone oil phase can be a solution of a solid organopolysiloxane gum or resin, or of a highly viscous organopolysiloxane gum, in a low viscosity organopolysiloxane fluid. The organopolysiloxane gum can for example have a viscosity of above 1000 cm²/s or even above 100000 cm²/s. The low viscosity organopolysiloxane fluid can for example have a viscosity in the range 1 to 1000 centiStokes (1 to 1000 mm²/s). The low viscosity organopolysiloxane fluid can be a cyclic polydiorganosiloxane such as decamethylcyclopentasiloxane and/or a linear polydiorganosiloxane such as a linear polydimethylsiloxane tipped with trimethylsiloxy units.

A reactive organopolysiloxane fluid can for example contain reactive groups such as hydroxyl (either Si—OH or alcohol groups), amino, vinyl or Si—H groups. The organopolysiloxane fluid can for example be a silanol-terminated polydimethylsiloxane. The reactive organopolysiloxane fluid may be mixed with a non-reactive organopolysiloxane fluid. An amino-functional organopolysiloxane is preferably mixed with at least 30%, more preferably at least 50%, of a non-reactive organopolysiloxane fluid such as a linear polydimethylsiloxane tipped with trimethylsiloxy units, based on the weight of the amino-functional organopolysiloxane.

The silicone oil phase is generally present at least 3% by volume of the emulsion and is usually present at least 10 or 20% to allow efficient distribution of the silicone emulsion. The silicone oil phase can form up to 90% by volume of the emulsion, more usually up to 85 or 86%. Preferred emulsions for easy incorporation into personal care products may contain 25 to 65% by volume silicone oil phase, for example 45 to 55% silicone oil phase.

We have found that a wide range of proteins and peptides are effective in stabilizing the silicone oil-in water-emulsion. The protein can for example be a milk-derived protein such as casein or whey protein. Vegetable proteins, in particular cereal proteins such as wheat protein (gluten) have also been found effective. Proteins derived from nuts such as almond or from other pulses, or soy protein, can also be effective. The protein can be in the form of a derivative such as a salt, for example casein can be in the form of sodium caseinate. A suitable sodium caseinate is sold under the trade mark lactalis'. The protein can be partially hydrolysed. Milk proteins are highly effective emulsion stabilizers without hydrolysis but vegetable proteins may be more effective in partially hydrolysed form. Examples of such hydrolysed proteins are partially hydrolysed gluten products sold by Tate & Lyle under the trade marks ‘Meripro 705’ and ‘Meripro 711’, and an almond-extracted protein hydrolysate sold by Cognis under the trade mark ‘Gluadin Almond’.

Not all proteins and peptides are effective in stabilizing the silicone oil-in water-emulsion. Proteins whose natural function is as structural proteins, such as keratin, the main structural protein of hair and wool, are generally not effective in emulsifying silicones. Plant proteins from plants where cellulose, not protein, is the main natural structural material usually effect emulsification of the silicone, as do proteins from natural emulsions such as milk proteins. The effectiveness of a candidate protein can be tested by mixing equal amounts of dimethicone and a 2% aqueous solution of the protein in a high shear mixer and observing whether an emulsion is formed or the dimethicone separates from the aqueous phase after mixing. If an emulsion is formed, and the mean particle size as well as 90^{th} percentile of the distribution are maintained for at least 2 weeks, the protein is effective, although improvements in the particle size and stability of the emulsion can probably be achieved by varying the proportions of materials or using a more sophisticated emulsifying apparatus.

Even the proteins which are effective in emulsifying the silicone may vary in the extent of stabilization of the emulsion over time. For example, the wheat protein hydrolysates ‘Meripro 705’ and the more extensively hydrolysed ‘Meripro 711’ both emulsify the silicone but the emulsions formed using ‘Meripro 705’ are more stable to long term storage. The emulsions formed using ‘Meripro 705’ are also more stable to long term storage than emulsions formed using ‘Gluadin Almond’. The silicone-in-oil emulsions formed using milk proteins such as casein generally have good long term stability.

Mixtures of proteins can be used to stabilize the emulsion, for example a mixture of proteins from different sources such as a milk protein with a vegetable protein or partially hydrolysed vegetable protein.

The amount of protein in the emulsion is generally at least 0.25% by weight of the emulsion to achieve stabilization of the emulsion and is preferably at least 0.5 or 0.75%. The amount of protein in the emulsion can be up to 20% by weight of the emulsion, and protein concentrations of 10 to 20% may give the lowest particle size emulsions, but protein concentrations of 0.5 to 7%, particularly 0.75 to 2.5%, are generally preferred and effectively stabilize silicone oil-in-water emulsions at median particle size in the range 1 to 30 μm. Generally the cumulative amount (e.g. weight:weight) of stabilizer(s) present will always be less than the amount of silicone oil, i.e. the protein or peptide is present in the emulsion in an amount by weight equal to or less than the silicone oil phase.

The emulsions of the invention can in general be produced by mixing the water, the silicone oil and the protein or peptide under high shear. Conveniently the protein or peptide can be dissolved in the water before mixing with the silicone oil. The high shear mixing apparatus can be any of those known for silicone oil-in-water production. For example the aqueous protein solution and silicone oil can be mixed in a rotor and stator mixing apparatus such as an ‘UltraTurrax’ (Trade Mark). Further mixing can be carried out if required in an apparatus applying increased shear to give a lower particle size emulsion, for example in a homogeniser, particularly a two stage pressure homogeniser such as a Rannie (Trade Mark) homogeniser, or microfluidiser, or a sonolator (ultrasonic mixer).

When preparing emulsions containing a high proportion of silicone oil phase, for example above 60% silicone, the silicone can be mixed with protein or peptide and a small amount of water under high mechanical shear to form a non-Newtonian “thick phase”, which has a very high viscosity at low shear rates (much more viscous at low shear rate than the silicone polymer alone). The high shear mixing in this case is carried out in a mixer designed to deal with thick pastes such as a dental mixer. On continued mixing the “thick phase” converts to an oil-in-water emulsion, which can be diluted with further water, optionally containing further protein or peptide, if required.

Emulsification can be carried out batchwise or continuously, for example the aqueous protein solution and silicone oil can be fed to a continuous emulsification apparatus such as that described in WO-02/42360-A2. In any mixing apparatus the mixing tool is preferably submerged in the aqueous silicone oil mixture to minimize entrapment of air and foam formation.

Emulsification is carried out at a temperature in the range 0 to 60° C., preferably 15 to 50° C. The emulsion should not be heated at over 60° C. during its production as high temperatures can denature the protein, that is irreversibly coagulate the protein.

The particle size of the emulsion depends on many factors such as the amount and type of protein emulsifier, the amount and type of silicone oil and the degree of shear applied during mixing. The median size of the silicone oil droplets in the emulsion is generally above 0.2 μm and usually above 0.4 μm, and can be up to 100 μm, but is preferably below 70 μm and usually below 30 or 50 μm.

The emulsion of the invention may contain a biocide, particularly a bactericide, to avoid bacterial growth in the emulsion. Bacterial growth can cause mould formation and can also cause degeneration of the protein or peptide which may reduce the long term stability of the emulsion. One example of a suitable bactericide is ‘Glycacil L’. (Trademark by Lonza). Bactericide can for example be present at 0.01 to 0.25% by weight of the emulsion.

It is generally preferred that the emulsion contains no (i.e. zero % by weight of protein or peptide) non-polymeric amphiphilic surfactant. Such conventional surfactants may compete with the protein emulsifier in an unfavourable manner. If any non-polymeric amphiphilic surfactant is present in the emulsion, it must be present at less than 25% by weight, preferably less than 10%, based on the weight of protein or peptide, and at less than 0.1% by weight, more preferably less than 0.02%, based on the weight of the emulsion.

The emulsion of the invention can contain one or more other additives known in silicone oil-in water emulsions, provided that the additive does not interact unfavourably with the protein and/or peptide. Solid additives can be present in minor amount; for example a fine hydrophobic silica can be mixed with a polydiorganosiloxane fluid before emulsifying when forming a silicone antifoam emulsion. Other additives which may be present include UV stabilizers, antioxidants, fragrances, emollients or pharmaceutical or cosmetic active materials. The need for polyhydroxy compounds (for example propylene glycol, sorbitol or glucose) is not envisaged in the present application but they may be added to the continuous (water) phase if necessary. However, preferably the present application does not include such polyhydroxy compounds.

### EXAMPLES

These examples are intended to illustrate the invention to one of ordinary skill in the art and should not be interpreted as limiting the scope of the invention set forth in the claims. All measurements and experiments were conducted at 23° C., unless indicated otherwise.

Parts and percentages are by weight unless indicated otherwise. Particle size measurements were made by laser diffraction technique using a “Mastersizer 2000” from Malvern Instruments Ltd., UK. Unless otherwise indicated all particle sizes indicated in the present application are mean average particle size according to D(v, 0.5).

### Example 1

3.59% ‘Meripro 705’ partially hydrolysed wheat protein was dissolved in 68.18% water. This solution was mixed with 28.23% dimethicone (trimethylsilyl-terminated polydimethylsiloxane) of viscosity 350 mm²/s (cSt) in an UltraTurrax high shear mixer. A stable emulsion was formed.

### Examples 2 to 13

In each of these Examples the partially hydrolysed wheat protein was dissolved in water and the resulting solution was mixed with silicone oil in a dental mixer (DAC 150 FZK SpeedMixer™ from Hauschild) dental mixer to form initially a thick paste which on further mixing became a stable viscous creamy emulsion. The proportions of ingredients used are shown in Table 1 below.

The partially hydrolysed wheat protein emulsifiers used were ‘Meripro 705’ and ‘Meripro 711’, which has a higher degree of hydrolysis. The silicone oils used were either of two dimethicones of different viscosities, or dimethiconol (silanol-terminated polydimethylsiloxane).

TABLE 1

| | | | Silicone | % | % | % |
|---|---|---|---|---|---|---|
| Example | Protein | Silicone | viscosity cSt | water | protein | silicone |
| 2 | Meripro 705 | Dimethicone | 350 | 13.56% | 0.71% | 85.73% |
| 3 | Meripro 705 | Dimethicone | 350 | 13.72% | 0.72% | 85.56% |
| 4 | Meripro 705 | Dimethicone | 350 | 13.36% | 0.70% | 85.94% |
| 5 | Meripro 705 | Dimethiconol | 1000 | 13.96% | 0.73% | 85.31% |
| 6 | Meripro 711 | Dimethiconol | 1000 | 13.98% | 0.74% | 85.28% |
| 7 | Meripro 711 | Dimethiconol | 1000 | 13.98% | 0.74% | 85.28% |
| 8 | Meripro 705 | Dimethiconol | 1000 | 13.49% | 0.71% | 85.80% |
| 9 | Meripro 705 | Dimethicone | 350 | 13.47% | 0.71% | 85.82% |
| 10 | Meripro 711 | Dimethiconol | 1000 | 13.73% | 0.72% | 85.55% |
| 11 | Meripro 711 | Dimethicone | 350 | 13.64% | 0.72% | 85.64% |
| 12 | Meripro 705 | Dimethicone | 350 | 13.50% | 0.71% | 85.79% |
| 13 | Meripro 711 | Dimethicone | 60000 | 13.40% | 0.71% | 85.90% |

### Example 14

1.0% casein sold under the Trade Mark ‘Emulac NA’ was dissolved in 50.0% water. This solution was mixed with 49.0% dimethiconol of viscosity 1000 cSt in an UltraTurrax high shear mixer to form an initial emulsion. The emulsion was then subjected to higher shear mixing in a two stage Rannie pressure homogeniser with the two stages operating at pressures of 700 and 360 bar. A stable emulsion was formed. The particle size was analysed and the median particle size by weight D05 and the particle size of the tenth largest percentile D01 and ninetieth largest percentile D09 are shown in Table 2 below.

### Examples 15 to 18

Following the procedure of Example 14 emulsions were prepared from aqueous solutions of ‘Emulac NA’ casein or of whey protein isolate sold under the Trade Mark ‘Globulan’ and dimethiconol or dimethicone silicone oils. The proportions of ingredients are shown in Table 2. Stable emulsions were produced of particle size shown in Table 2

TABLE 2

| | | | Silicone | | | | | | |
|---|---|---|---|---|---|---|---|---|---|
| | | | Viscosity | % | % | % | D01 | D05 | D09 |
| Example | Protein | Silicone | cSt | water | protein | silicone | μm | μm | μm |
| 14 | Casein | Dimethiconol | 1000 | 39.00% | 1.00% | 60.00% | 0.888 | 3.439 | 9.942 |
| 15 | Globulan | Dimethiconol | 1000 | 39.00% | 1.00% | 60.00% | 1.168 | 4.473 | 8.86 |
| 16 | Casein | Dimethicone | 350 | 72.50% | 2.50% | 25.00% | 0.732 | 1.589 | 5.16 |
| 17 | Casein | Dimethicone | 350 | 73.75% | 1.25% | 25.00% | 0.225 | 1.375 | 4.283 |
| 18 | Casein | Dimethicone | 350 | 74.50% | 0.50% | 25.00% | 0.869 | 1.953 | 4.7 |

The storage stability of the emulsions of Examples 14 to 18 were tested. 0.25% ‘Glycacil L’ bactericide was added to each emulsion and the emulsions were stored in a sealed container at ambient temperature. Each of the emulsions appeared stable for at least 400 days. The particle size of the emulsions was tested early in the storage test and again after at least 400 days. The results are shown in Table 3 below.

TABLE 3

| Example | Age in days | D01 μm | D05 μm | D09 μm |
|---|---|---|---|---|
| 14 | 42 | 0.929 | 3.737 | 8.237 |
| 14 | >400 | 0.624 | 2.471 | 8.254 |
| 15 | 42 | 1.127 | 4.201 | 8.341 |
| 15 | >400 | 0.177 | 2.643 | 8.688 |
| 16 | 8 | 0.643 | 1.531 | 5.31 |
| 16 | >400 | 0.245 | 1.608 | 5.62 |
| 17 | 33 | 0.444 | 1.483 | 4.208 |
| 17 | >400 | 0.209 | 1.752 | 5.62 |
| 18 | 33 | 0.858 | 1.843 | 4.399 |
| 18 | >400 | 0.209 | 1.752 | 5.62 |

### Example 19

2.0% casein was dissolved in 48.0% water. This solution was mixed with 50.0% silicone in an UltraTurrax high shear mixer to form a silicone oil-in-water emulsion. The silicone was a 12% solution of an ultrahigh viscosity dimethiconol gum in dimethicone sold under the Trade Mark Dow Corning DC1503′ and having viscosity 1500 mm²/s. The particle size was analysed and the results are shown in Table 4 below.

### Examples 20 to 22

The emulsion of Example 19 was subjected to higher shear mixing in a high pressure homogeniser operating at pressures of 200, 400 or 700 bar. The particle size of each resulting emulsion was analysed and the results are shown in Table 4 below.

### Examples 23 to 26

Examples 19 to 22 were repeated replacing the Dow Corning DC1503′ by a 15% solution of the ultrahigh viscosity dimethiconol gum in decamethylcyclopentasiloxane (cyclomethicone) sold under the Trade Mark Dow Corning DC1501′ and having viscosity 6000 mm²/s. The particle size of each resulting emulsion was analysed and the results are shown in Table 4 below.

TABLE 4

| | | | % | % | % | Emulsifying | D01 | D05 | D09 |
|---|---|---|---|---|---|---|---|---|---|
| Example | Protein | Silicone | water | protein | silicone | apparatus | μm | μm | μm |
| 19 | Casein | Gum + | 48.00% | 2.00% | 50.00% | Ultraturrax | 1.478 | 12.545 | 48.885 |
| | | dimethicone | | | | | | | |
| 20 | Casein | Gum + | 48.00% | 2.00% | 50.00% | homogeniser | 1.848 | 15.446 | 59.924 |
| | | dimethicone | | | | −200 bar | | | |
| 21 | Casein | Gum + | 48.00% | 2.00% | 50.00% | homogeniser | 1.802 | 16.211 | 68.306 |
| | | dimethicone | | | | −400 bar | | | |
| 22 | Casein | Gum + | 48.00% | 2.00% | 50.00% | homogeniser | 1.196 | 7.865 | 44.321 |
| | | dimethicone | | | | −700 bar | | | |
| 23 | Casein | Gum + | 48.00% | 2.00% | 50.00% | Ultraturrax | 1.409 | 12.516 | 45.168 |
| | | cyclomethicone | | | | | | | |
| 24 | Casein | Gum + | 48.00% | 2.00% | 50.00% | homogeniser | 1.316 | 10.57 | 42.798 |
| | | cyclomethicone | | | | −200 bar | | | |
| 25 | Casein | Gum + | 48.00% | 2.00% | 50.00% | homogeniser | 1.251 | 9.241 | 33.574 |
| | | cyclomethicone | | | | −400 bar | | | |
| 26 | Casein | Gum + | 48.00% | 2.00% | 50.00% | homogeniser | 1.147 | 7.966 | 33.149 |
| | | cyclomethicone | | | | −700 bar | | | |

### Examples 27 to 29

Various concentrations of ‘Meripro 705’ partially hydrolysed wheat protein were dissolved in water and mixed with dimethicone of viscosity 350 cSt in the proportions shown in Table 4 below in an UltraTurrax high shear mixer. Each emulsion was subjected to higher shear mixing in a high pressure homogeniser operating at 700 bar. The particle size of each resulting emulsion was analysed and the results are shown in Table 5 below.

TABLE 5

| | | | Silicone | % | % | % | D01 | D05 | D09 |
|---|---|---|---|---|---|---|---|---|---|
| Example | Protein | Silicone | viscosity | water | protein | silicone | μm | μm | μm |
| 27 | Meripro | Dimethicone | 350 | 74.50% | 0.5% | 25.00% | 0.865 | 1.954 | 5.401 |
| | 705 | | | | | | | | |
| 28 | Meripro | Dimethicone | 350 | 73.75% | 1.25% | 25.00% | 0.183 | 1.33 | 3.901 |
| | 705 | | | | | | | | |
| 29 | Meripro | Dimethicone | 350 | 72.50% | 2.5% | 25.00% | 0.122 | 1.101 | 4.106 |
| | 705 | | | | | | | | |

0.25% ‘Glycacil L’ bactericide was added to each of the emulsions of Examples 27 to 29 and the emulsions were stored and tested as described for Examples 14 to 18 above. Each of the emulsions appeared stable for at least 400 days. The particle size results are shown in Table 6 below.

TABLE 6

| Example | Age in days | D01 μm | D05 μm | D09 μm |
|---|---|---|---|---|
| 27 | 7 | 0.898 | 2.062 | 5.244 |
| 28 | 7 | 0.144 | 1.242 | 3.909 |
| 29 | 7 | 0.19 | 1.213 | 3.413 |
| 27 | 410 | 0.56 | 1.72 | 4.11 |
| 28 | 410 | 0.213 | 1.421 | 3.228 |
| 29 | 410 | 0.232 | 0.988 | 2.819 |

### Example 30

2.5% ‘Meripro 705’ partially hydrolysed wheat protein was dissolved in 47.5% water. 25% of a diaminofunctional organopolysiloxane of viscosity 0.8 to 5.0 mm²/s sold under the Trade Mark Dow Corning 8040′ was dissolved in 25% dimethicone of viscosity 1000 cSt. (mm²/s) The two solutions were mixed in an UltraTurrax high shear mixer to form a silicone oil-in-water emulsion. The particle size was analysed and the results are shown in Table 7 below.

### Examples 31 to 33

The emulsion prepared in Example 30 was subjected to higher shear mixing in a high pressure homogeniser operating at various pressures. The particle size of each resulting emulsion was analysed and the results are shown in Table 7.

### Examples 34 to 39

Further emulsions were prepared from the solution of ‘Meripro 705’ used in Example 30 and mixtures of Dow Corning 8040′ diaminofunctional organopolysiloxane and dimethicone of viscosity 1000 cSt in various proportions as shown in Table 7. The emulsification process was either the UltraTurrax mixer as used in Example 30 or the high pressure homogeniser as used in Examples 31 to 33 and is indicated in Table 7. The particle size of each resulting emulsion was analysed and the results are shown in Table 7.

### Examples 40 to 43

Examples 30 to 33 were repeated replacing the Dow Corning 8040′ diaminofunctional organopolysiloxane by a different aminofunctional organopolysiloxane of viscosity 3500 mm²/s. sold under the Trade Mark Dow Corning 8630′. The particle size of each resulting emulsion was analysed and the results are shown in Table 7.

TABLE 7

| | | | | % | | | | |
|---|---|---|---|---|---|---|---|---|
| | % | % | % | Amino- | Emulsifying | D01 | D05 | D09 |
| Example | water | protein | Dimethicone | silicone | apparatus | μm | μm | μm |
| 30 | 47.5 | 2.5 | 25.0 | 25.0 | UltraTurrax | 1.75 | 8.465 | 23.155 |
| 31 | 47.5 | 2.5 | 25.0 | 25.0 | homogeniser | 1.743 | 6.186 | 18.138 |
| | | | | | 200 bar | | | |
| 32 | 47.5 | 2.5 | 25.0 | 25.0 | homogeniser | 1.33 | 5.097 | 12.217 |
| | | | | | 400 bar | | | |
| 33 | 47.5 | 2.5 | 25.0 | 25.0 | homogeniser | 0.969 | 3.216 | 8.296 |
| | | | | | 700 bar | | | |
| 34 | 47.5 | 2.5 | 37.5 | 12.5 | homogeniser | 3.739 | 7.918 | 16.207 |
| | | | | | 700 bar | | | |
| 35 | 47.5 | 2.5 | 37.5 | 12.5 | homogeniser | 2.729 | 5.684 | 10.968 |
| | | | | | 360 bar | | | |
| 36 | 47.5 | 2.5 | 17.5 | 32.5 | homogeniser | 2.878 | 6.858 | 14.207 |
| | | | | | 700 bar | | | |
| 37 | 47.5 | 2.5 | 17.5 | 32.5 | homogeniser | 3.821 | 10.371 | 25.815 |
| | | | | | 360 bar | | | |
| 38 | 47.5 | 2.5 | 12.6 | 37.4 | UltraTurrax | 4.63 | 22.936 | 51.368 |
| 39 | 47.5 | 2.5 | 12.6 | 37.4 | homogeniser | 3.915 | 10.583 | 26.96 |
| | | | | | 700 bar | | | |
| 40 | 47.5 | 2.5 | 25.0 | 25.0 | UltraTurrax | 2.918 | 5.849 | 11.245 |
| 41 | 47.5 | 2.5 | 25.0 | 25.0 | homogeniser | 1.565 | 6.028 | 13.186 |
| | | | | | 200 bar | | | |
| 42 | 47.5 | 2.5 | 25.0 | 25.0 | homogeniser | 1.01 | 4.831 | 9.974 |
| | | | | | 400 bar | | | |
| 43 | 47.5 | 2.5 | 25.0 | 25.0 | homogeniser | 0.874 | 3.198 | 7.219 |
| | | | | | 700 bar | | | |

### Example 44

6.24% lactalis' sodium caseinate was dissolved in 43.76% water and was mixed with 50.0% dimethicone of viscosity 1000 cSt in an UltraTurrax mixer, then in three consecutive homogenization runs operated at 700 bar. A stable emulsion was formed of median particle size (D05) 0.683 μm.

### Examples 45 and 46

Following the process of Example 1, emulsions of high silicone concentration were prepared using ‘Emulac NA’ casein as emulsifier. Stable emulsions were formed which could be diluted with care. The formulations used and median particle size of the emulsions produced are shown in Table 8 below

TABLE 8

| | | Silicone | % | % | % | |
|---|---|---|---|---|---|---|
| Example | Silicone | viscosity | water | protein | silicone | D05 μm |
| 45 | Dimethicone | 1000 cSt | 14 | 1 | 85 | 45.83 |
| 46 | Dimethicone | 60000 cSt | 14 | 1 | 85 | 73.6 |

### Examples 47-50

1.25% Sodium caseinate and 1.25% whey protein were dissolved in 47.43% water and 0.07% Glycacil L was added. This solution was them mixed with 50% dimethicone of 350 cSt, using different mixing devices and pressure. The formulations' mean size is given in Table 9

TABLE 9

| | | D01 | D05 | D09 |
|---|---|---|---|---|
| Example | homogenization | [um] | [um] | [um] |
| 47 | UltraTurrax | 5.146 | 15.62 | 33.367 |
| 48 | Homogenizer, 240/110 bars | 0.27 | 3.044 | 8.756 |
| 49 | Homogenizer, 410/220 bars | 0.128 | 1.825 | 6.637 |
| 50 | Homogenizer, 700/340 bars | 0.146 | 1.167 | 3.512 |

### Examples 51-54

1.25% Sodium caseinate (Lactalis) and 1.25% Meripro 705 were dissolved in 47.42% water and 0.08% Glycacil L was added. This solution was them mixed with 50% dimethicone of 350 cSt, using different mixing devices and pressure. The formulations' mean size is given in Table 10

TABLE 10

| | | D01 | D05 | D09 |
|---|---|---|---|---|
| Example | homogenization | [um] | [um] | [um] |
| 51 | UltraTurrax | 0.229 | 9.263 | 24.057 |
| 52 | Homogenizer, 220/110 bars | 0.146 | 1.967 | 6.395 |
| 53 | Homogenizer, 420/280 bars | 0.16 | 1.476 | 5.139 |
| 54 | Homogenizer, 680/360 bars | 0.185 | 0.869 | 2.239 |

### Example 55-58

1.25% whey protein and 1.25% Meripro 705 were dissolved in 47.41% water and 0.09% Glycacil L was added. This solution was them mixed with 50% dimethicone of 350 cSt, using different mixing devices and pressure. The formulations' mean size is given in Table 11.

TABLE 11

| | | D01 | D05 | D09 |
|---|---|---|---|---|
| Example | homogenization | [um] | [um] | [um] |
| 55 | UltraTurrax | 4.509 | 11.139 | 36.344 |
| 56 | Homogenizer, 200/90 bars | 0.223 | 4.782 | 10.44 |
| 57 | Homogenizer, 400/220 bars | 0.169 | 2.38 | 6.651 |
| 58 | Homogenizer, 750/370 bars | 0.207 | 1.162 | 3.104 |

1. A silicone oil-in-water emulsion comprising 3 to 90% by volume of a silicone oil phase, characterized in that the emulsion is stabilized by a protein or peptide present at 0.25 to 20% by weight of the emulsion and that the emulsion contains less than 25% by weight of non-polymeric amphiphilic surfactant based on the weight of protein or peptide.

2. The emulsion according to claim 1 characterised in that the protein is casein and/or a derivative of casein.

3. The emulsion according to claim 1 characterised in that the protein is whey protein.

4. The emulsion according to claim 1 characterised in that the protein is wheat protein.

5. The emulsion according to claim 1I characterised in that the protein is partially hydrolysed.

6. The emulsion according to claim 1 characterised in that the protein comprises a mixture of proteins from different sources.

7. The emulsion according to claim 1 characterised in that the protein is present at 0.5 to 7% by weight of the emulsion.

8. The emulsion according to claim 1 characterised in that the silicone oil phase comprises a polydiorganosiloxane oil of viscosity 5 to 60000 centiStokes

9. The emulsion according to claim 1 characterised in that the silicone oil phase comprises a solution of an organopolysiloxane gum in a fluid polydiorganosiloxane of viscosity 1 to 1000 centiStokes.

10. The emulsion according to claim 8 characterised in that the silicone oil phase comprises a mixture of an amino-functional organopolysiloxane with a polydiorganosiloxane oil containing no organic functional groups.

11. The emulsion according to claim 1 characterised in that the silicone oil phase is present at 25 to 65% by volume of the emulsion.

12. The emulsion according to claim 1 characterised in that the emulsion contains a bactericide.

13. The emulsion according to claim 1 characterised in that the emulsion contains zero % non-polymeric amphiphilic surfactant based on the weight of protein or peptide.

14. The emulsion according to claim 1 characterised in that the protein or peptide is present in the emulsion in an amount by weight equal to or less than the silicone oil phase.

15. A process for the preparation of an emulsion according to claim 1 characterised in that the water, the silicone oil, and the protein or peptide are mechanically mixed under high shear at a temperature in the range 0 to 60° C.

16. The process according to claim 15 characterised in that an aqueous solution of the protein or peptide is mixed with the silicone oil.

17. The emulsion according to claim 1 characterised in that the protein is partially hydrolysed and is selected from the group consisting of casein and/or a derivative of casein, whey protein, wheat protein, and combinations thereof, and wherein the silicone oil phase comprises a silicone selected from the group consisting of dimethicone, dimethiconol, dimethiconol gum, cyclomethicone, amino-functional organopolysiloxane, and combinations thereof.
